(19) United States
(12) Patent Application Publication
(10) Pub. No.: US 2005/0182084 A1
(43) Pub. Date: Aug. 18, 2005

(54) Method for treating erectile dysfunction

(75) Inventors: Takayuki Yamaji, Osaka (JP); Shunji Yamazaki, Osaka (JP); Raymond Price, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJISAWA PHARMACEUTICAL CO., LTD., Osaka (JP)

(21) Appl. No.: 11/038,194

(22) Filed: Jan. 21, 2005

Related U.S. Application Data
(60) Provisional application No. 60/610,539, filed on Sep. 17, 2004.
(60) Provisional application No. 60/537,014, filed on Jan. 20, 2004.

(51) Int. Cl. A61K 31/4745

(52) U.S. Cl. 514/291

(57) ABSTRACT

Macrolide compound (I) is provided for treating or preventing erectile dysfunction, which is, for example, induced by or secondary to diseases, alcoholism, aging, arterial insufficiency, venous leakage, hormonal insufficiency, drug use, surgery, chemotherapy or radiation, particularly, for preventing or treating an erectile dysfunction of mammals.

### TECHNICAL FIELD

This invention relates to a medical use of a macrolide compound for treating or preventing erectile dysfunction.

### BACKGROUND ART

WO02/07757 shows a method for treating or preventing male erectile dysfunction or female sexual arousal disorder by administering an effective amount of vascular endothelial growth factor (VEGF), brain-derived neurotrophic factor (BDNF), basic fibroblast growth factor (bFGF), etc.

WO02/096420 shows a use of some compounds for treating or preventing nerve injury caused as a consequence of prostate surgery.

WO96/40140 shows that a certain pipecolic acid derivative having an affinity for FKBP-type immunophilins, such as tacrolimus, stimulate growth of damaged peripheral nerves or promote neuronal regeneration.

WO02/053159 shows a neurotrophic activity of a compound (I) mentioned below.

### DISCLOSURE OF INVENTION

The inventors of this invention have found that the compound (I), mentioned below, has an excellent activity for treating or preventing erectile dysfunction, which is induced, for example, by or secondary to diseases, alcoholism, aging, arterial insufficiency, venous leakage, hormonal insufficiency, drug use, surgery, chemotherapy or radiation.

Accordingly, this invention provides a new use of the compound (I) for treating or preventing erectile dysfunction.

Further, this invention provides a method for preventing or treating erectile dysfunction.

The macrolide compound used in the present invention has the following chemical formula. 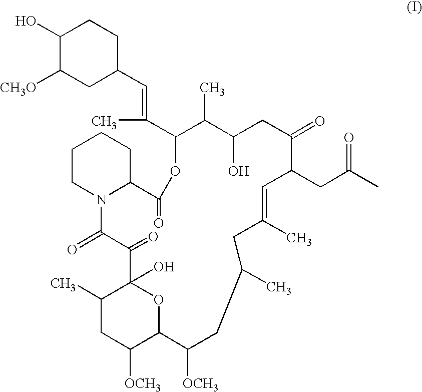
It has already been produced in U.S. Pat. No. 5,376,663, example 29.

With respect to the compound (I) used in the present invention, it is to be understood that there may be conformers and one or more stereoisomers such as optical and geometrical isomers due to asymmetric carbon atom(s) or double bond(s), and such conformers and isomers are also included within the scope of the compound in the present invention. The most preferable compound (I) is the following compound (Ia).

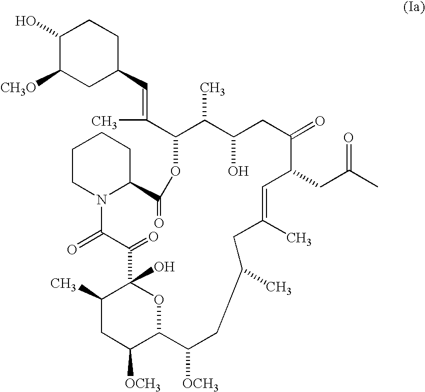

And further, the compound(I) can be in the form of a pharmaceutically acceptable salt, derivatives, solvate or pro-drug, which is included within the scope of the present invention. The solvate preferably include a hydrate and an ethanolate.

The compound (I) in the present invention may be administered as a pure compound or a mixture with other compounds, preferably, in a pharmaceutical vehicle or carrier.

The compound (I) in this invention can be used in the form of a pharmaceutical preparation, for example, in solid, semisolid or liquid form, which contains the compound(I), as an active ingredient, in admixture with an organic or inorganic carrier or excipient suitable for external(topical), enteral, intravenous, intramuscular, or parenteral applications. The active ingredient may be compounded, for example, with the usual non-toxic, pharmaceutically acceptable, carriers for tablets, pellets, capsules, eye drops, suppositories, solutions (saline, for example), emulsion, suspensions (olive oil, for example), ointment, aerosol sprays, cream, skin plasters, patches and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, or liquid form, and in addition auxiliary, stabilizing, thickening and coloring agents and perfumes may be used. The active object compound is included in the pharmaceutical composition in an effective amount sufficient to produce the desired effect upon the process or condition of the disease.

Mammals which may be treated using the method of the present invention include livestock mammals such as cows, horses, etc., domestic animals such as dogs, cats, rats, etc. and humans.

While the dosage of therapeutically effective amount of the compound (I) varies from and also depends upon the age and condition of each individual patient to be treated, a daily dose of about 0.0001-1000 mg, preferably 0.001-500 mg and more preferably 0.01-100 mg. of the active ingredient is generally given for treating diseases, and an average single dose of about 0.001-0.01 mg, 0.2-0.5 mg, 1 mg, 5 mg, 10 mg, 50 mg, 100 mg, 250 mg and 500 mg is generally administered. Daily doses for chronic administration in humans will be in the range of about 0.1-30 mg/kg/day.

And further, the compound (I) can be applied, simultaneously, separately or sequentially, with other agents having an activity for treating or preventing erectile dysfunction.

The following examples illustrate the present invention in further detail. It should be understood that those examples are not intended to limit the scope of the invention.

### EXAMPLE 1

The solution of the compound (Ia) comprising the following ingredients was prepared by dissolving the compound (Ia) and HCO-60 in ethyl alcohol by a conventional manner.

| | | |
|---|---|---|
| Compound (Ia) | 1 | mg |
| HCO-60 | 400 | mg |
| (Polyoxyethylenehydrogenated | | |
| castor oil 60) | | |
| Ethyl alcohol | to 1 | ml |

### EXAMPLE 2

Effect of the compound (Ia) on erectile dysfunction was confirmed by a rat cavernous nerve injury model.

Method:

(1) The recovery of erectile function after cavernous nerve injury was basically assessed in a similar manner to that of BJU International 92, 470-475 (2003).
(2) The compound (Ia), in a form of the solution which was prepared in a similar manner to that of Example 1 mentioned above, or its placebo was given to rats, from 1 day after surgery to 1 day before harvesting for 8 weeks, subcutaneously after being diluted with a suitable amount of HCO-60/EtOH in physiological saline.
   Result:

The effect of the compound (Ia) on intracavernous pressure is shown in the following Table 1.

TABLE 1

| | Peak intracavernous |
|---|---|
| | pressure(cm H₂O) |
| | mean ± S.E. |
| Sham (n = 6) | 134.0 ± 5.6 |
| Vehicle-treated (n = 7) | 34.5 ± 10.2### |
| Compound (Ia) (n = 7) | 97.4 ± 8.0** |
| 1 mg/kg | |

###P < 0.001, v.s. sham (Dunnett's post-test)
**P < 0.01, v.s. vehicle-treated(Dunnett's post-test)

The above test result shows that the compound (Ia) has a remarkable recovery effect on intracavernous pressure(cm H₂O) Therefore, the above result indicates that the compound (I), particularly the compound (Ia), is useful for treating or preventing erectile dysfunction. The erectile dysfunction may be induced by or secondary to diseases, alcoholism, aging, arterial insufficiency, venous leakage, hormonal insufficiency, drug use, surgery, chemotherapy or radiation.

Particularly, the compound (I) is useful for treating or preventing erectile dysfunction induced by or secondary to diabetes.

And further, the erectile dysfunction induced by or secondary to surgery, such as a prostate surgery, can be exemplified as a particular one. More particularly, erectile dysfunction caused as a consequence of prostate surgery, for example, erectile dysfunction caused by an injury to a penile cavernous nerve of mammals, can be exemplified.

The present invention further provides methods for treating or preventing erectile dysfunction, which is, for example, induced by or secondary to diseases, alcoholism, aging, arterial insufficiency, venous leakage, hormonal insufficiency, drug use, surgery, chemotherapy or radiation, which comprises administering an effective amount of the compound (I) to mammals.

According to the invention, the compound (I) may be administered systemically.

The present invention further provides the following ones. A commercial package comprising the pharmaceutical composition containing the compound (I) identified in the above and a written matter associated therewith, wherein the written matter states that the compound (I) can or should be used for preventing or treating erectile dysfunction, which is, for example, induced by or secondary to diseases, alcoholism, aging, arterial insufficiency, venous leakage, hormonal insufficiency, drug use, surgery, chemotherapy or radiation.

The patents, patent applications and publications cited herein are incorporated by reference.

1: A method of manufacturing a medicament for treating or preventing erectile dysfunction comprising mixing a compound of formula (I): 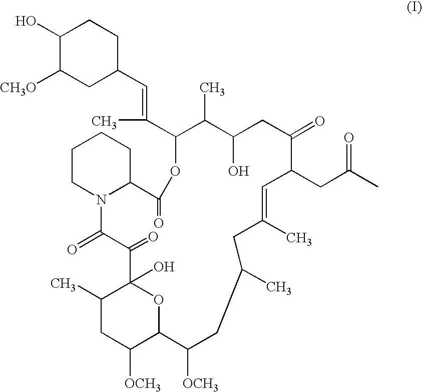
with a pharmaceutically acceptable vehicle or carrier.

2: The method of claim 9, wherein the erectile dysfunction is induced by or secondary to a disease, alcoholism, aging, arterial insufficiency, venous leakage, hormonal insufficiency, drug use, surgery, chemotherapy or radiation.

3: The method of claim 2, wherein the erectile dysfunction is induced by or secondary to diabetes.

4: The method of claim 3, wherein the diabetes is diabetic neuropathy.

5: The method of claim 2, wherein the erectile dysfunction is induced by or secondary to surgery.

6: The method of claim 5, wherein the surgery is prostate surgery.

7: The method of claim 2, wherein the erectile dysfunction is caused by an injury to a penile cavernous nerve.

8: The method of claim 9, wherein the compound of formula (I) is a stereoisomer represented by formula (Ia):

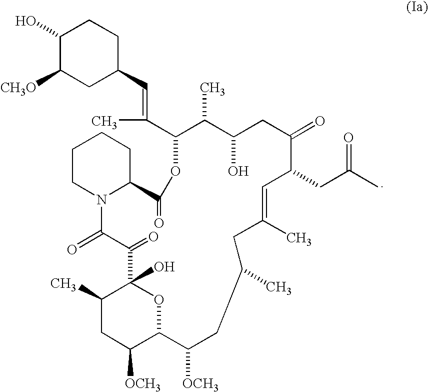

9: A method for preventing or treating erectile dysfunction in a subject in need thereof, which comprises administering to said subject an effective amount of a composition comprising a compound of formula (1):

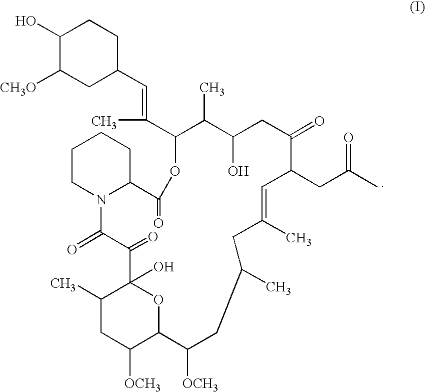

10: (canceled)

11: A medicament made by the method of claim 1, wherein the compound of formula (I) is in a form of its pharmaceutically acceptable salt, derivative, pro-drug or solvate.

12: A commercial package comprising a medicament made by the method of claim 1 and a written matter associated therewith, wherein the written matter states that the compound (I) can or should be used for preventing or treating erectile dysfunction.

13: The medicament of claim 11, wherein the compound of formula (I) is in a solvate form selected from the group consisting of a hydrate and an ethanolate.

14: The method of claim 9, wherein the compound of formula (I) is in a form of its pharmaceutically acceptable salt, derivative, pro-drug or solvate.

15: The method of claim 9, wherein the compound of formula (I) is in a solid, semisolid or liquid form.

16: The method of claim 9, wherein the compound of formula (I) is administered at a daily dose ranging from 0.0001-1000 mg.

17: The method of claim 9, wherein the compound of formula (I) is administered at a daily dose ranging from 0.001-500 mg.

18: The method of claim 9, wherein the compound of formula (I) is administered at a daily dose ranging from 0.01-100 mg.

19: The method of claim 9, wherein the compound of formula (I) is administered chronically at a daily dose ranging from about 0. 1-30 mg/kg/day.

20: The method of claim 9, wherein said subject is selected from the group consisting of a cow, a horse, a dog, a cat, a rat, and a human.

21: The method of claim 9, wherein said subject is a human.
